(19) United States
(12) Patent Application Publication
(10) Pub. No.: US 2026/0284389 A1
(43) Pub. Date: Sep. 24, 2026

(54) SYSTEMS AND METHODS FOR MULTICHANNEL INDIVIDUALIZED STIMULATION THERAPY

(71) Applicant: THE UNITED STATES OF AMERICA, AS REPRESENTED BY THE SECRETARY, DEPARTMENT OF HEALTH AND HUMAN, Bethesda, MD (US)

(72) Inventors: Zhi-De DENG, Bethesda, MD (US); Bruce Allen PRITCHARD, Portland, OR (US); Junghee KIM, Germantown, MD (US); George Raphael DOLD, Boyds, MD (US); Robert Hyllel SCHOR, Kensington, MD (US); Sarah Hollingsworth LISANBY, Durham, NC (US)

(21) Appl. No.: 19/474,167

(22) PCT Filed: Apr. 10, 2024
(86) PCT No.: PCT/US2024/023876

§ 371 Date: Oct. 9, 2025

Related U.S. Application Data
(60) Provisional application No. 63/495,244, filed on Apr. 10, 2023.

(51) Int. Cl. A61N 1/36; A61N 1/02; A61N 1/04

(52) U.S. Cl. CPC A61N 1/36034; A61N 1/025; A61N 1/0456; A61N 1/0476; A61N 1/0484; A61N 1/36025; A61N 1/36031

(57) ABSTRACT

Methods and systems are provided herein for a multichannel individualized stimulation therapy (MIST) system. In one example, a MIST system includes a device comprising a plurality of stimulation electrodes and a plurality of isolated current sources (ICS), wherein the device is reconfigurable to vary positions and dosimetry of pulses of stimuli applied to a patient.

### CROSS REFERENCE TO RELATED APPLICATIONS

The present application is a U.S. National Phase of International Application No. PCT/US2024/23876 entitled “SYSTEMS AND METHODS FOR MULTICHANNEL INDIVIDUALIZED STIMULATION THERAPY,” and filed on Apr. 10, 2024. International Application No. PCT/US2023/079114 claims priority to U.S. Provisional Application No. 63/495,244 Entitled “systems and Methods for Multichannel Individualized STIMULATION THERAPY”, filed Apr. 10, 2023. The entire contents of each of the above-listed applications are hereby incorporated by reference for all purposes.

### TECHNICAL FIELD

Embodiments of the subject matter disclosed herein relate to medical treatment, and more specifically to methods and system for multichannel individualized stimulation therapy.

### BACKGROUND AND SUMMARY

Electroconvulsive therapy (ECT) is a commonly used treatment option for medication-resistant depression and/or other mental health disorders. In ECT, a generalized tonic-clonic seizure is elicited by inducing an electric field in the brain through delivery of current to electrodes placed on the scalp. ECT functions by delivering a series of brief pulses of current through a pair of electrodes affixed to a scalp of an anesthetized patient. Present clinical ECT practice uses limited fixed electrode placements, typically bitemporal or right unilateral, and fixed high current amplitudes, such as 800 or 900 mA that induce strong electric fields. The electric fields are broadly distributed throughout the brain and the expected maximal effect of the generated ECT stimulus train is of those regions of the brain in proximity to the placement of the two electrodes. While stimulating large portions of the brain in this way may have powerful anti-depressant effects, it may also result in significant cognitive side effects, such as memory loss. These cognitive side effects may be due to overstimulation of the medial temporal brain regions.

Optimization of ECT is impeded by lack of a stimulation paradigm and technology to optimally target brain structures mediating the anti-depressant action of ECT while avoiding stimulation of regions responsible for cognitive side effects. Furthermore, conventional fixed current amplitude stimulation dosing may fail to account for inter-individual anatomical variability in patients. Variability in anatomy from patient to patient may produce inconsistent dosage when a fixed current amplitude is used, which may result in variable clinical outcomes. Systems and methods are therefore needed to provide technology for maximizing antidepressant efficacy of ECT while minimizing side effects.

In one example, the issues described above may be at least partially addressed by a multichannel individualized stimulation therapy (MIST) system. The MIST system herein disclosed includes a multi-channel, multi-electrode design coupled with dense electroencephalogram (EEG) recording for real-time seizure topography monitoring and closed-loop seizure shaping.

The MIST system may comprise, for example, a plurality of independently programmable current sources delivered by a multi-electrode cap affixed to a patient's head. The MIST system may be capable of delivering a full range of adjustable stimulation parameters with pulse amplitude up to maximum current, such as 900 mA. User-defined stimulation protocols, based on multi-electrode optimization methods and computational electric field modeling and optimization algorithms, may serve as inputs to the system. The multi-electrode optimization algorithm may use individual neuroimaging data to construct patient-specific head models and/or to determine electrode configuration for targeted stimulation. Further, each isolated current source designated for the particular neurostimulation session may have a defined current in mA, with a total amount of current delivered by all isolated current sources designated for the particular neurostimulation session being a sum of the currents of each isolated current source. In this way, positions of electrodes and dosimetry of current applied through each of the electrodes is reconfigurable for each patient.

In some examples, the MIST system herein disclosed comprises a main control unit (MCU), a power control unit (PCU), and a plurality of isolated current sources (ICSs). Each source is isolated from the other current sources, with each current source driven by a separate isolated battery. Isolation of the current sources and their respective batteries may reduce influence from neighboring current sources. The multi-electrode cap that is connected to the isolated current sources is interfaced with a patient. The multi-electrode cap comprises a plurality of stimulation electrodes that are interleaved with high-density EEG electrodes to provide an ECT stimulus train that encompasses a larger active area, either via simultaneous activation of spatially-isolated brain regions or activation of a spatially-diffuse area. In this way, an individualized ECT stimulus train may be generated, with variable electrode placements based on a patient's anatomy. The individualized ECT stimulus train may introduce current to specified brain areas that allows for reduction in cognitive side effects by avoiding areas of the brain that may result in neurocognitive side effects if stimulated.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows an example multichannel individualized stimulation (MIST) system, according to an embodiment;

FIG. 2 shows an example H-bridge circuit topology and resulting waveform of the MIST system of FIG. 1;

FIG. 3 shows a flowchart illustrating a method for configuring the MIST system;

FIG. 4 shows a flowchart illustrating a method for operating the MIST system; and

FIG. 5 shows example stimulations from the MIST system with a multi-electrode configuration in comparison to conventional electroconvulsive therapy (ECT) system stimulations with bitemporal and right unilateral electrode configurations.

### DETAILED DESCRIPTION

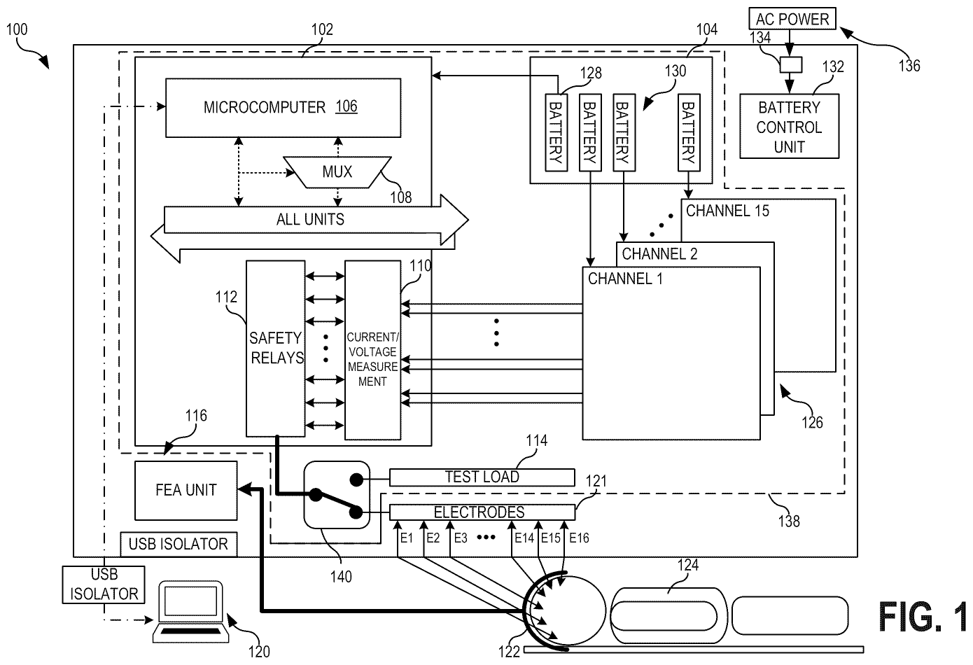

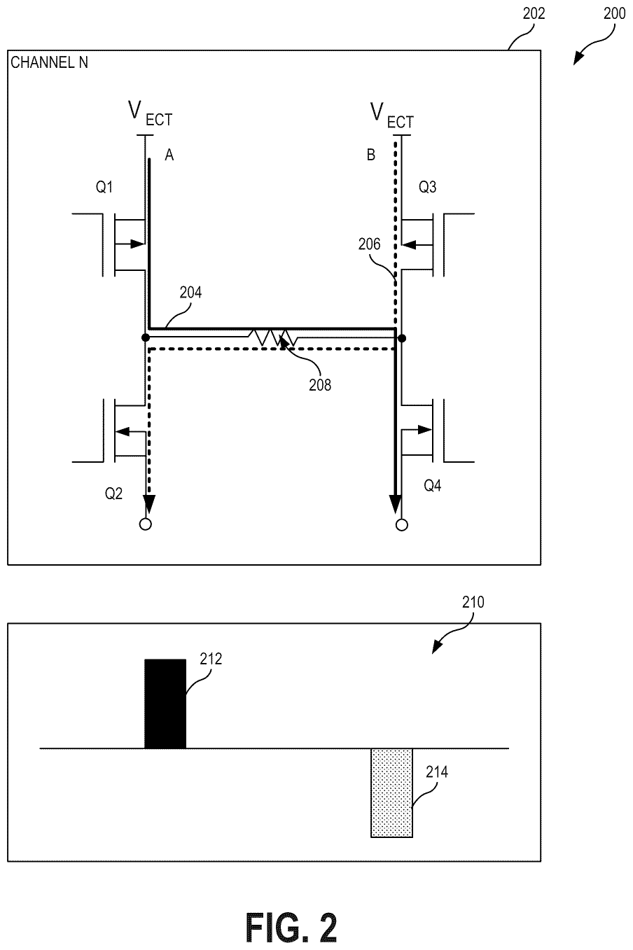

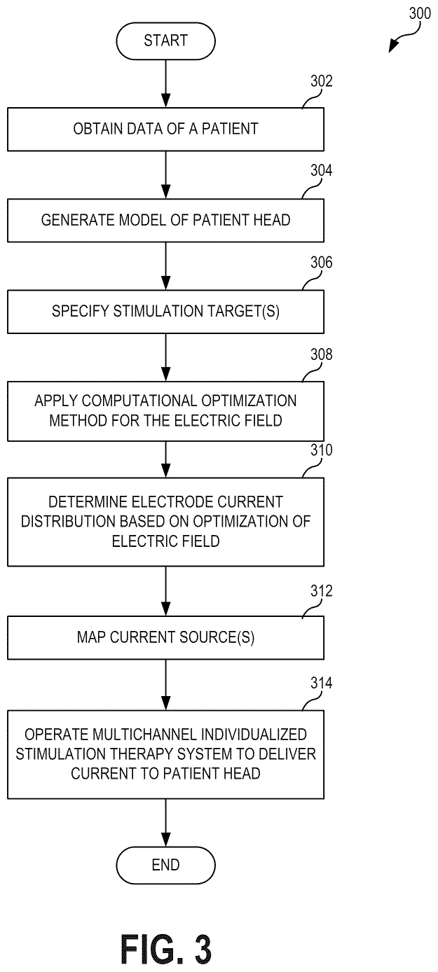

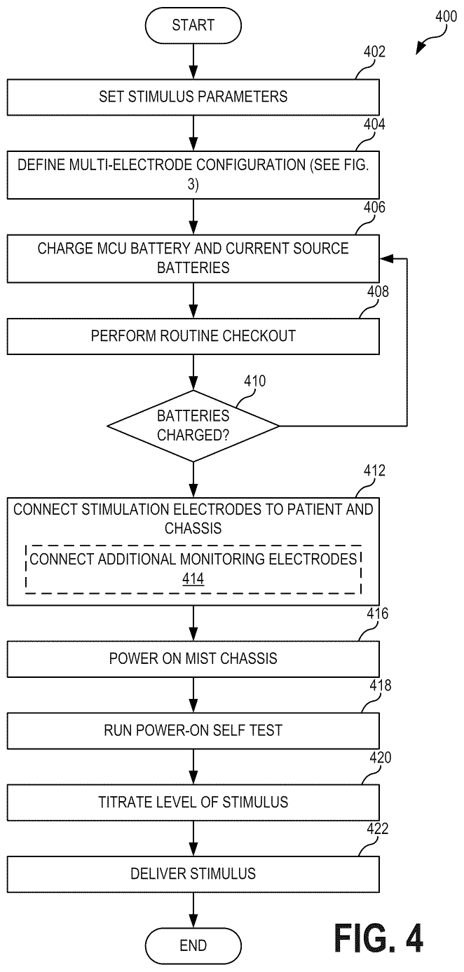

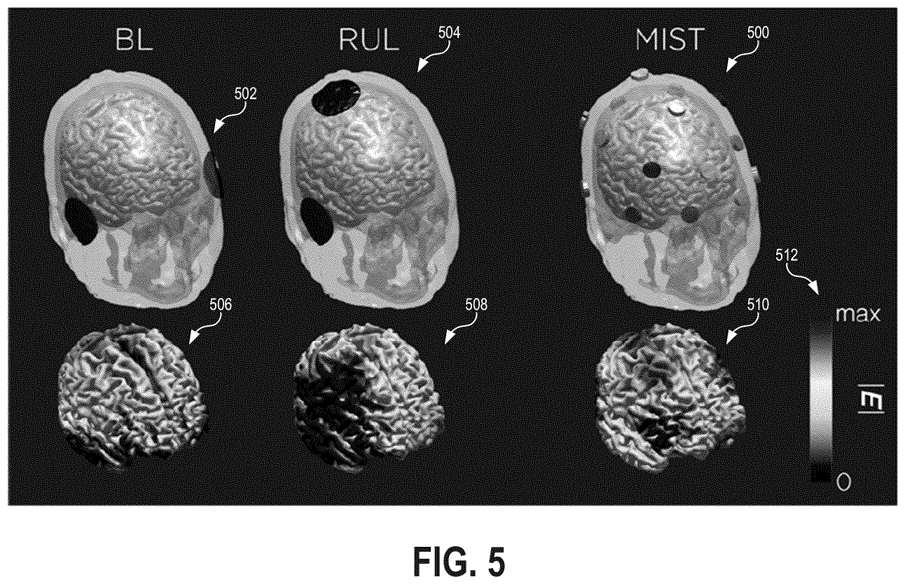

The following description relates to systems and methods for a multichannel individualized stimulation therapy (MIST) system comprising a main control unit (MCU) comprising a microcomputer, a plurality of isolated current sources (ICSs) each powered by an isolated battery source, and a multi-electrode cap comprising a plurality of electrodes through which current is delivered while a front-end amplifier unit (FEAU) reads physiology signals, for example electroencephalogram (EEG), electrocardiogram (ECG), and electromyogram (EMG) signals. The MIST system herein described may be designed to deliver multichannel electrical stimulation to patients being treated for various neurological, psychological, and psychiatric disorders by electroconvulsive therapy (ECT). A diagram of the MIST system is shown in FIG. 1. An example of current output of an isolated current source is shown in FIG. 2. A method for configuring the MIST system, including configuration of electrodes and distributions of current is shown in a flowchart in FIG. 3. A method for operating the MIST system is shown in a flowchart in FIG. 4. An example of stimulation to brain tissue via the MIST system in comparison to conventional ECT stimulations is shown in FIG. 5.

Turning now to FIG. 1, a high-level block diagram of a MIST system 100 is shown. The MIST system 100 comprises an MCU 102, a PCU 104, and a plurality of ICSs 126. In some examples, the MIST system 100 may include, for example, up to fifteen isolated current sources. The MCU 102, PCU 104, and plurality of ICSs 126 may be configured as part of and/or within a chassis 138.

The MCU 102 may be operated by a microcontroller or microcomputer 106 that includes a field programmable gate array, a processor (e.g., a single-core or dual-core processor), one or more indicator light-emitting diodes (LEDs), and multiple analog and digital input/output lines. In some examples, the MCU 102 may be WiFi-capable. In other examples, the MCU 102 may be include wired communications. The microcomputer 106 may control the PCU 104 and the plurality of ICSs 126 through a serial peripheral interface (SPI) protocol and/or the field programmable gate array. The MCU 102 may control current path of each of a plurality of electrodes by controlling position of a plurality of switches of the plurality of ICSs 126 and the MCU 102. Control of position of each of the plurality of switches may be reconfigurable, based on determination of electrode configuration and assignment of current sources to pairs of electrodes, as will be further described below.

The MCU 102 further comprises a multiplexer 108, current/voltage measurements 110, safety relays 112, and a test load 114. The microcomputer 106 may be connected to the multiplexer 108. The multiplexer 108 may select between several analog and/or digital input signals and may forward the selected signals to other components of the MIST system 100. The multiplexer 108 may allow for several connections over a single channel. As such, the multiplexer 108 may be selectively connected to the current/voltage measurements 110, the safety relays 112, the FEAU 116, an operator computer 120, the plurality of ICSs 126, and the PCU 104 via an SPI protocol. The microcomputer 106 may receive the current/voltage measurements 110 via the multiplexer 108 during stimulation on a pulse-by-pulse basis. Further, the microcomputer 106 may detect potential anomalies in real-time (e.g., without intentional time delay) and may abort a stimulation pulse train in the event of detected current amplitude or latency deviations.

The microcomputer 106 may send digital bits via the multiplexer 108 to activate gate drivers in one or more current sources, as well as to the safety relays 112 to allow stimulus delivery to the patient 124 via a multi-electrode cap 122. During stimulation, the plurality of ICSs 126 are controlled by the field programmable gate array of the MCU 102, which may achieve precise and accurate timing of delivery. The plurality of ICSs 126, as controlled by the MCU 102, may produce electrical stimulation pulses. The current/voltage measurements 110 may be coupled to the safety relays 112 and to the plurality of ICSs 126 via separate communication busses.

The safety relays 112 may be coupled to either the test load 114 or to the multi-electrode cap 122 via a load control switch 140 (e.g., a changeover switch). Depending on a position of the load control switch 140, e.g., coupled to either the multi-electrode cap 122 or to the test load 114, stimulation pulses may be delivered to either the patient 124 via the multi-electrode cap 122 or to the test load 114. The test load 114 may allow for testing of stimulating current path and the accuracy of the stimulus current whereby the current is provided by each of the plurality of the ICSs 126. Delivering stimulus current to the test load 114 may ensure that the proper function of MIST.

Activated gate drivers in one or more of the plurality of ICSs 126 may result in delivered stimulus to the patient 124 when the safety relays 112 allow passage of current to the multi-electrode cap 122.

The multi-electrode cap 122 may include a plurality of stimulation electrodes interleaved with one or more EEG electrodes (e.g., high-density EEG electrodes). An electrode box 121 may include one or more inputs and one or more outputs, wherein electrode cables from the multi-electrode cap 122 are connected to the electrode box 121 and a connection cable from the electrode box 121 is connected to the MCU 102.

In some examples, the multi-electrode cap 122 comprises up to fifteen stimulation electrodes. Which of the fifteen stimulation electrodes are used for a respective neurostimulation session may be determined based on an electrode current distribution that is determined by a computational electric field optimization method, as will be described below. A sixteenth electrode may also be included in the MIST system 100, serving as a reference electrode to estimate electrical impedance of the fifteen stimulating electrodes. Estimation may be accomplished by sending a single small bi-phasic current pulse between the reference electrode and each of the stimulation electrodes being used for a respective neurostimulation session. Estimation of electrical impedance may allow for determination of sufficient contact between the electrodes and the head of the patient 124. In some examples, a conduction gel may be applied between the cap and the patient's head. In conjunction with detection of potential anomalies (e.g., deviations in actual stimulation current compared to expected current), the estimation of electrical impedance may decrease inaccuracies of delivered stimulus and allow for abortion of a stimulus train in the event of inaccurate delivered stimulus.

Each of the plurality of ICSs 126 may provide circuitry and power to deliver a pulse train stimulus to one pair of stimulation electrodes of the multi-electrode cap 122. No two or more current sources may deliver stimulus pulses to the same pair of stimulation electrodes. One electrode may be included in more than one pair of electrodes. For example, a first electrode and a second electrode may form a first pair of electrodes. The first electrode and a third electrode may form a second pair of electrodes. A first current source may deliver stimulus to the first pair and a second current source may deliver stimulus to the second pair. Definition of the pairs and determination of which current sources deliver stimulus to which of the pairs may be provided by user input or generated by one or more algorithms, as will be described with respect to FIG. 3.

In addition to generating the stimuli, the plurality of ICSs 126 may monitor voltage present on each pair of electrodes as well as monitoring current flowing during the pulse stimuli via the current/voltage measurements 110. For example, the plurality of ICSs 126 may monitor accuracy of ICS current, while the MCU 102 monitors total energy that is delivered to a patient. In response to the total energy meeting and/or exceeding a predefined threshold, stimulus delivery is aborted.

A range of possible currents for stimulation may be defined, the range being from 1 mA to 900 mA, in some examples, and currents for each of one or more current sources designated for a neurostimulation session may be reconfigurable and/or adjustable based on the computed electric field and/or electrode configuration specific to the patient. A stimulus generated by the plurality of ICS 126 may be a square constant-current pulse characterized by rapid onset and offset, a relatively constant current when on, and the same current for every pulse in the train. As the stimulus is presented, some area of a patient's head between stimulating electrodes (e.g., source electrode) may experience the high current intensity that is higher than any of the plurality of ICSs 126 is assigned to deliver, due to the position of the electrodes and the direction of the current between each pair of stimulating electrodes from the multi-electrode cap 122. To deliver the current from each of the plurality of ICSs 126 in this condition, they may provide higher voltage that can accommodate the total current during stimulation.

The stimulus delivered may be bi-phasic, including a positive and a negative phase, as will be described further with respect to FIG. 2.

The microcomputer 106 is further communicatively coupled to the operator computer 120. A connection between the microcomputer 106 and the operator computer 120 may be an electrically isolated wired connection (e.g., a USB cable) or a wireless connection (e.g., over Wi-Fi). The operator computer 120 may be a desktop computer, laptop computer, mobile device, and/or the like that includes a display device through which a graphical user interface (GUI) may be displayed. The GUI of the operator computer 120 may allow for a user to define stimulation parameters, enable stimulus delivery, and receive feedback on completion status of the stimulus train, battery health, and other device status indications. The stimulation parameters that may be defined by the user may include current dosimetry, stimulus train duration, pulse polarity, pulse width (e.g., duration of each current pulse), and/or pulse-pair frequency. In some examples, one or more of the parameters may be determined based on a computer implemented algorithm.

The operator computer 120 may be further communicatively and/or operably coupled to the FEAU 116, for example via a wired connection such as a USB. The FEAU 116 may comprise an EEG protective circuit and an EEG amplifier/reader. In this way, the FEAU 116 may monitor signals, such as EEG, ECG, and/or EMG signals, during the neurostimulation session. The FEAU 116 may also protect EEG electrodes during stimulation and reduce EEG reading recovery time delay. The FEAU 116 may be communicatively and/or operably coupled to the multi-electrode cap 122 that is positioned in direct contact with the patient 124 (e.g., the patient's head). The operator computer 120 and/or a separate display device communicatively coupled to the FEAU 116 may display an EEG reading as provided by the EEG electrodes of multi-electrode cap 122, and the FEAU 116. The operator computer 120 may store data recording settings and parameters of a neurostimulation session including state and status of software running on both the operator computer 120 and on the MCU 102, and recorded timing, voltages, and currents measured by the MCU 102 for each delivered stimulus pulse.

In some examples, the MIST system 100 has one or more modes of operation including a charge mode and a stimulation mode. In charge mode, alternating current (AC) power 136 may provide power to the MIST system 100. The AC power 136 may be converted into direct current (DC) via an AC/DC converter 134. Power may be provided to a battery control unit (BCU) 132 which is operably coupled to the PCU 104. The PCU 104 comprises an MCU battery 128 and a plurality of ICS batteries 130. The BCU 132 may be operably coupled to both the MCU battery 128 and to each of the plurality of ICS batteries 130 to charge each of the batteries when in charging mode. In some examples, the MCU battery 128 and each of the plurality of ICS batteries 130 are rechargeable batteries such as nickel metal hydride batteries.

The plurality of ICS batteries 130 may include a battery for each of the plurality of ICSs 126, wherein each of the plurality of ICSs 126 is coupled to only one of the plurality of ICS batteries 130. The current sources batteries may be isolated from each other, in some examples. Isolation of the plurality of ICSs 126 from each other and isolation of the plurality of ICS batteries 130 from each other may allow for delivery of current to brain areas via a pair of electrodes without being influenced by neighboring current sources. Further, isolation of ICS batteries and current sources may minimize noise, thereby ensuring that the MIST system 100 operates with minimal interference and noise-related issues.

In stimulation mode, the MIST system 100 may be battery operated. The BCU 132 may be disconnected from the PCU 104 when in stimulation mode. In some examples, the chassis 138 may include a mechanical guard that ensures that when stimulation electrode cables are plugged in, the battery charging connector (e.g., the wired connection between the BCU 132 and the PCU 104) is disconnected. Operating with battery power alone during a neurostimulation session may decrease possibility of patient to Earth ground shortage. For example, the PCU 104 may be configured to isolate the patient from any conductive surfaces of hardware of the MIST system 100 and electrodes to Earth ground. As noted, the multi-electrode cap 122 may include a plurality of stimulation electrodes that are interleaved with high-density EEG electrodes. The microcomputer 106 may provide control for the EEG protective circuit of the FEAU 116 to protect the EEG amplifier during stimulus current delivery. The EEG data may be sent to the operator computer 120 for signal digitation and display, as previously discussed. Alternatively or additionally, a chart recorder for visual monitoring of the EEG seizure during the treatment session may be displayed by the operator computer 120 and/or by a separate display device not connected to the MCU 102.

The MIST system 100 may consider the finite limitations of energy and charge that can be safely delivered to a patient during stimulation. While the predicted charge value may not differ significantly from the measured value, the energy delivered may vary during stimulation as the energy calculated is based on a bulk resistor model that assumes a fixed resistance of 220 Ohms. The fixed resistance may not always be accurate given the complex geometry of the patient's head and the variable placement of the electrodes. To obtain a better estimate of the delivered energy, the MIST system 100 may utilize two techniques: pre-stimulation and two-stage stimulation monitoring. Pre-stimulation involves the ICSs 126 delivering a small amplitude current pulse prior to the high amplitude stimulation to estimate electrode-to-skin impedances. The two-stage stimulation monitoring evaluates the current and voltage measurements from the current sources and the electrodes; wherein the plurality of the ICSs 126 measures the current and voltage from the source side and the MCU measures the current and voltage from the electrodes side. The stimulation may be terminated immediately if the stimulation current measurements from the plurality of ICSs 126 deviates from expected and/or the total accumulated energy exceeds a predefined threshold. Pulses, as herein described, may be short bursts of current and are not meant to specify the type of current, for example pulses may or may not be applied with DC bias.

Turning now to FIG. 2, an example of an ICS 202 of the MIST system 100 is shown. The ICS 202 may be one of the plurality of ICSs 126 of FIG. 1. The ICS 202, and other ICSs of the MIST system 100, may be configured as an H-bridge circuit to deliver current to the patient's head via the multi-electrode cap 122 or to the test load 114. The ICS 202 may have two output terminals, A and B. As discussed, stimuli delivered via the ICS 202 are biphasic, wherein one of the A and B terminals serves as a voltage source and the other of the terminals serves as a current sink. As such, the H-bridge circuit topology may include a positive phase 204 and a negative phase 206 of a pulse, both depicted in a circuit 200 of FIG. 2. In some examples, each of the terminals A and B may be assigned to an electrode of an electrode pair, thereby defining a path for the current pulse to travel. As will be described below, designation of electrode pairs and assignment of current sources to electrode pairs may be reconfigurable based on patient-specific anatomy and one or more stimulation targets. In other examples, such as when the MIST system is configured for impedance tomography, terminals of an ICS may be connected to more than one electrode at a time.

During the positive phase 204 of the pulse, depicted by a solid line in FIG. 2, switches Q1, Q2, Q3, and Q4 may be in a first configuration. The first configuration may include switches Q1 and Q4 being closed while switches Q2 and Q3 are open. The first configuration may allow current to flow across a load 208 in a first direction. The load 208 may be the head of the patient 124 or the test load 114 depending on a position of the load control switch 140 as described with reference to FIG. 1. During the negative phase 206 of the pulse, depicted by a dotted line in FIG. 2, switches Q1, Q2, Q3, and Q4 may be in a second configuration. The second configuration may include switches Q2 and Q3 being closed while switches Q1 and Q4 are open. The second configuration may allow current to flow in a second direction opposite the first direction across the load 208.

A resultant waveform 210 is also shown in FIG. 2. A first pulse 212 may correspond to the positive phase 204 of the pulse and is consequently depicted as a positive pulse. A second pulse 214 may correspond to the negative phase 206 of the pulse and is consequently depicted as a negative pulse. The first pulse 212 and the second pulse 214 may form a bi-phasic pulse. Multiple bi-phasic pulses may be delivered by the ICS 202 during a neurostimulation session.

During setup of the neurostimulation session, a configuration of electrodes and current sources may be defined, indicating sites of the head of the patient to be stimulated and which electrodes are to send current to/through which sites. From the defined configuration, assignment of terminals and prescribed delivered current for each isolated current source in use may be generated. A total current prescribed for the neurostimulation session may be a sum of each of the prescribed delivered currents (each specific to one of the ICSs). In some examples the sum of each of the prescribed delivered currents may be equal to or less than 900 mA. As an example, the plurality of ICSs may produce source voltage that can accommodate a total accumulated stimulus current that is formed in a path of between electrodes, whereby a high current carrying path is due to position of electrodes and direction of the current between each pair of active electrodes of the multi-electrode cap.

In some examples, a titration loading scheme may be employed during a neurostimulation session. The titration loading scheme may include gradually increasing current doses that allows for determination of the lowest demanded dose for seizure induction, as will be described with reference to FIG. 4.

Referring now to FIG. 3, a flowchart illustrating an example method 300 for configuring a MIST system, such as MIST system 100 described with respect to FIG. 1, which includes a plurality of ICSs and a plurality of electrodes of a multi-electrode cap. Method 300 may be carried out according to instructions stored in memory of one or more controllers, processors, and/or computing devices included as part of and/or communicatively or operatively coupled to the MIST system, for example microcomputer 106 of MCU 102 of MIST system 100 described with reference to FIG. 1.

At 302, method 300 includes obtaining data of a patient. The data may include structural data, such as magnetic resonance imaging (MRI) data, of the patient's brain and/or brain mapping data, such as EEG data. The data of the patient may define patient-specific anatomy that may be used to generate a model of the patient's anatomy. In some examples, structural data may be segmented and then meshed. For example, brain MRI data may be segmented into scalp, skull, cerebrospinal fluid, gray matter, and white matter.

At 304, method 300 includes generating a three-dimensional (3D) model of the patient's head based on the segmented data. As noted, each of the segmented portions of the data may be meshed together to form the 3D model of the patient's head. The 3D model may be configured for use in one or more methods and/or algorithms. For example, the 3D model may be used to compute an electric field and current flow that is to be provided by the MIST system during a neurostimulation session. Computation of the electric field and/or current flow may be performed via application of a numerical simulation model, such as a finite element method or a boundary element method. Computing the electric field via the numerical stimulation model may decrease time spent by the operator in determining the electric field and/or current flow. Computation of the electric field and/or current flow based on patient-specific anatomy may allow for individualized therapy.

At 306, method 300 includes specifying stimulation target(s). Specification may be performed by a user of the MIST system via an operator computer that is communicatively and/or operatively coupled to the MIST system. Determination of the stimulation target(s) may be based on the data of the patient, for example brain mapping data such as an EEG that may provide information for which areas of the patient's brain are to be targeted during the neurostimulation session. Further, specification of stimulation target(s) may allow for avoidance of certain brain areas that contribute to side effects like memory loss and as such specifying stimulation targets that do not include those areas may allow for decreased side effects for the patient. In addition to specifying targets, one or more non-targets (e.g., anti-targets) that are not to receive stimulation may also be specified, in some examples.

At 308, method 300 includes applying a computational optimization method for the electric field. In some examples, the computational optimization method may be applied in order to determine current distribution demanded to achieve maximum electric field delivery to the specified stimulation targets. The computational optimization method may be carried out by instructions stored in non-transitory memory of the MIST system.

At 310, method 300 includes determining electrode current distribution based on the computational optimization method. As noted, the computational optimization method may allow for determination of the electrode current distribution. In some examples, the electrode current distribution may include positions of each of the electrodes as well as amount of current in mA to be delivered to and/or through the positions. In this way, the electrode current distribution may be individualized to the patient as the computational optimization method is applied based on the 3D model of the patient's head.

The multi-electrode cap may comprise the plurality of electrodes in fixed positions, though what part of the electrode delivers or receives current may be defined for specified sites. For example, a first electrode may be assigned to a first site and a first current amount may be assigned to the first site, while a second electrode may be assigned to a second site and a second current amount may be assigned to the second site. Each of the current amounts may be a positive or negative number. An example optimization method applied at 308 may define current amounts with a minimum variance, such that a range between the highest amount of delivered current and the lowest amount of delivered current is as small as possible.

At 312, method 300 includes mapping current sources of the MIST system. Mapping current sources may include assigning current sources to electrode pairs and determining current produced by each of the current sources. In some examples, a number of current sources of the plurality of ICSs that are defined for use by the mapping may be less than a total number of current sources in the plurality of ICSs. For example, as described with reference to FIG. 1, the plurality of ICSs may comprise 15 current sources. A number of defined electrodes in use, as known based on the specified stimulation target(s) and electrode current distribution, may be N. A corresponding number of current sources for the neurostimulation session may be N−1.

As is described with respect to FIG. 2, each of the plurality of ICSs may comprise two output terminals, A and B, and stimuli may be delivered as pairs of alternating positive and negative current pulses. In the mapping of current sources, electrodes are defined as pairs, with one electrode of a pair being assigned to one of A and B terminals of a corresponding current source and the other electrode of the pair being assigned to the other of A and B terminals. In this way, based on electric field optimization, defined positions of electrodes, pairs of electrodes, and assignments of current source, multiple paths of stimulus may be defined that stimulate the specified stimulation target(s).

In some examples, mapping of current sources may be constrained by one or more situations. For example, certain current sources may be used for certain sites of the patient's brain, and/or certain current sources may be assigned based on the currents determined by the electric field optimization algorithm. As another example, preferred electrode pairs may be assigned to a current source. Further, in some examples, a power-on self test run by the MIST system prior to delivering stimuli may reveal issues with one or more current sources.

At 314, method 300 includes operating the MIST system to deliver current to the patient's head based on the configuration and currents determined at 312. As will be further described with respect to FIG. 4, the MIST system may be charged, connected to the multi-electrode cap, and powered on prior to delivery of the stimuli through the brain.

Turning now to FIG. 4, a flowchart illustrating an example 400 for operating a MIST system is shown. The MIST system herein described may be the MIST system 100 of FIG. 1. Method 400 may be carried out at least in part according to instructions stored in memory of one or more controllers, processors, and/or computing devices included as part of and/or communicatively or operatively coupled to the MIST system, for example microcomputer 106 of MCU 102 of MIST system 100 described with reference to FIG. 1. The MIST system may comprise a chassis that includes an MCU, PCU, and a plurality of ICSs, and FEAU, an operator computer removably coupled to the chassis via an isolated wired connection, and a multi-electrode cap removably coupled to the chassis.

At 402, method 400 includes setting stimulus parameters. As described with reference to method 300, one or more parameters of the stimulus may be defined by a user or by an algorithm. The one or more parameters may include pulse polarity (e.g., direction of current flow), pulse width (e.g., duration of each current pulse), pulse-pair frequency (e.g., pulse-pairs per second), train duration (e.g., duration of pulse-pair train), and/or total current flowing during a pulse (e.g., up to 900 mA) may be defined by the user via user inputs to the operator computer or via an algorithm (e.g., the computational optimization method or a separate algorithm).

At 404, method 400 includes determining a multi-electrode configuration. As described in detail with reference to FIG. 3, structural data (e.g., MRI data) of a patient may be obtained and segmented. From the segmented structural data, a 3D head model of the patient may be generated and the 3D head model may be used to define electric field and current flow. Stimulation targets of the patient's brain may be specified, in some examples based brain mapping data (e.g., EEG data). A computational optimization method may be used to determine electrode current distribution for the specified stimulation target(s). Given this electrode current distribution, current source mapping may be performed to define electrode pairs, assign current sources (e.g., channel boards) to electrode pairs, and assign terminals of each current source to electrodes of corresponding electrode pairs.

At 406, method 400 includes charging an MCU battery and a plurality of ICS batteries. As described with reference to FIG. 1, the MIST system may be operated in charging mode or stimulation mode. In charging mode, AC power is converted to DC power and used to charge the MCU battery and the plurality of ICS batteries. Each of the plurality of ICS batteries may be connected to one of the plurality of ICSs.

At 408, method 400 includes performing a routine checkout. Performing the routine checkout may comprise powering on the chassis in order to run a power-on self test. The power-on self test may determine whether each of the batteries, including the MCU battery and the plurality of ICS batteries, are fully charged. The power-on self test may also include determining functional status of each of the isolated current sources, as will be further described below.

At 410, method 400 judges whether each of the batteries is fully charged, as based on results of the routine checkout. If the routine checkout determines that the batteries are fully charged, the method 400 proceeds to 412. If the routine checkout determines that the batteries are not fully charged, the method 400 returns to 406 to continue charging the batteries.

At 412, method 400 includes connecting stimulation electrodes to the patient (e.g., connecting the multi-electrode cap to the patient's head, including placement of connection gel between cap and scalp) and to the chassis. In some examples, the stimulation electrodes may be connected to an electrode box which may then be connected to the chassis via an isolated cable/wire. Positions of the electrodes for the multi-electrode cap may be defined based on the configuration determined at 404. In some examples, connection between the electrodes and the chassis may be performed manually. Additionally, interleaved EEG electrodes may be included in the multi-electrode cap, as discussed with reference to FIG. 1. Connection of the cap to the patient's head may also connect the EEG electrodes to the patient's head for EEG monitoring.

In some examples, additional monitoring electrodes may also be connected to the patient and to the chassis in a similar fashion, as noted at 414. These additional monitoring electrodes may include EMG electrodes and EKG electrodes. During a neurostimulation session, the patient may be temporarily paralyzed, for example as a result of a neuromuscular-blocking agent. Monitoring EMG electrodes may monitor for adequate paralysis during the neurostimulation session and monitoring EKG electrodes may monitor cardiac status during the neurostimulation session.

At 416, method 400 includes powering on the MIST chassis. In some examples, powering on the chassis may be performed manually via a switch or button on an exterior of the chassis. The operator computer may be connected to the MCU via an isolated wired connection or via WiFi. In some examples, the switch or button, when in the ON position may enable the battery connection to be connected and while in the OFF position may prohibit the battery connection from being connected. The operator computer may also be powered on and in operation. When the MIST chassis is powered on, including powering on the MCU therein, a connection between the operator computer and the MCU may be established.

At 418, method 400 includes running a power-on self test. The power-on self test may be run in response to the operator computer establishing the connection between the operator computer and the MCU. As described above, the power-on self test verifies a plurality of statuses, including battery health and charge and status of each of the plurality of isolated current sources. Verifying the status of each of the plurality of isolated current sources may comprise verifying that each can measure current and voltage, generate pulse trains, and measure impedance of test loads using very brief low amplitude pulses. If any of the status verifications do not pass, a notification may be sent to the user via the operator computer or may be provided by the chassis (e.g., via an LED indicator). In addition, in response to failure of the power-on self test, the MIST system may be shut down, and automatically be powered off.

Further, in response to passing each of the status verifications, the user may upload the MIST configuration, determined at 404, of electrode pairs, current source assignments, and current amounts, thereby setting parameters for each of the current sources in use for the neurostimulation session.

At 420, method 400 includes titrating stimulus levels. As is discussed above, stimuli generated during the neurostimulation session may induce a seizure within the patient. Titrating the stimulus levels may enable determination of what threshold level of stimulus is required to induce the seizure for the patient. The threshold level of stimulus may vary patient to patient, and as such titration may further allow for individualization of stimulation. Titration may comprise delivering a stimulus that is predicted to be sub-threshold, and then sequentially delivering increased amounts of stimuli following a predetermined protocol until a seizure is induced. Once a seizure threshold is determined based on the titration, a treatment level of stimulus that is relative to the threshold level of stimulus may be determined.

At 422, method 400 includes delivering stimulus to the patient. One or more parameters, as discussed at 402, may define frequency, duration, and amplitude of pulses of the stimulus. The stimulus may include multiple channels of current from current sources to multiple pairs of electrodes, as determined at 404, allowing for delivery of stimulus to multiple targets within the brain, if so desired and defined during configuration of the MIST. Delivery of stimulus may be in response to user input (e.g., activation of one or more buttons on the chassis). Outputs and/or results of the neurostimulation session may be recorded and displayed by the operator computer, including voltages, currents, and other parameters of each current source/electrode pair. Further, current and voltage parameters expected for each pulse in a pulse train and for each stimulating electrode are recorded, whereby the ICSs measure intended current from each of the ICSs, and the MCU measures cumulative stimulus current between electrodes on a patient's head. In some examples, in response to current and voltage deviating from expected, the pulse train is immediately aborted and the neurostimulation session is ended.

Further, a separate EEG recording may be displayed by the operator computer on a display device. The EEG recording may be saved to disk memory for access at later times. Recording EEG waveforms during the neurostimulation session may allow for real-time seizure topography monitoring and closed-loop seizure shaping.

Once the stimulus has been delivered, the MIST system may enter an idle state in which the MIST system is powered on, but no actions are in progress (e.g., no stimulus is being delivered). In the idle state, battery and MCU checks may be run periodically (e.g., every 5 seconds) to verify status of the batteries and the microcomputer (and other components of the MCU). Following 422, method 400 ends.

Turning now to FIG. 5, an example MIST system multi-electrode configuration 500 of the multi-electrode cap 122 of FIG. 1 is shown. Resultant stimulation of the brain from the multi-electrode cap 122 is shown as well. Stimulation via the MIST system 100 is shown in comparison to conventional ECT electrode placements, including a bitemporal configuration 502 and a right unilateral configuration 504.

A stimulation level key 512 is provided in FIG. 5. A first stimulation pattern 506 resulting from the bitemporal configuration 502 of electrodes is shown. Increased stimulation is shown in areas of a current path between the electrodes of the bitemporal configuration (e.g., in the temporal lobes and across the frontal lobe of the brain) and decreased stimulation is shown in areas outside of the current path (e.g., over the parietal and occipital lobes). Similarly, a second stimulation pattern 508 resulting from the right unilateral configuration 504 of electrodes is shown. Increased stimulation is shown in areas of a current path between the electrodes (e.g., over the right temporoparietal region of the brain) while decreased stimulation is shown in areas outside of the current patient (e.g., over the left parietal region of the brain).

In contrast to the first and second stimulation patterns 506, 508, a third stimulation pattern 510 resulting from the multi-electrode configuration 500 is shown. Increased stimulation is shown in various regions of the brain as a result of multiple current paths passing through the brain from a plurality of pairs of electrodes.

In some examples, the areas of desired increased stimulation of the third stimulation pattern 510 may be defined based on the patient's anatomy (as mapped by imaging, EEG readings, or other means as discussed above), targets of interest within the brain, and the like. Parameters such as these may be used to define electrode positions, current paths, pulse directions (negative vs positive), and current amplitudes. The multi-electrode cap herein described may have the electrodes therein in fixed positions, but defined current positions (inputted by a user or defined by an algorithm) may indicate which electrodes provide or receive current (e.g., which electrodes are assigned to which terminals of indicated ICSs), thereby allow for flexibility in producing focal and individualized stimulation to brain regions.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A system, comprising:
a device comprising a plurality of stimulation electrodes and a plurality of isolated current sources (ICSs), wherein the device is reconfigurable to vary positions and dosimetry of pulses of stimuli applied to a patient.

2. The system of claim 1, further comprising:
a main control unit (MCU) comprising a microcomputer;
a power control unit (PCU) comprising a plurality of batteries including an MCU battery and a plurality of ICS batteries, wherein each of the plurality of ICSs is powered by one of the plurality of ICS batteries and is isolated from each of the other of the plurality of ICSs; and
a multi-electrode cap comprising the plurality of stimulation electrodes, wherein one or more of the plurality of ICSs delivers current through pairs of stimulation electrodes of the multi-electrode cap.

3. The system of claim 2, wherein each of the one or more of the plurality of ICSs is assigned to a pair of stimulation electrodes of the multi-electrode cap based on a configuration of the multi-electrode cap.

4. The system of claim 3, wherein the configuration of the multi-electrode cap is determined based on patient-specific anatomy and one or more stimulation targets.

5. The system of claim 2, wherein the multi-electrode cap further comprises one or more electroencephalogram (EEG) electrodes interleaved with the plurality of stimulation electrodes.

6. The system of claim 2, further comprising an operator computer communicatively and operatively coupled to the MCU.

7. The system of claim 6, wherein the operator computer displays a graphical user interface (GUI) through which one or more stimulus parameters may be defined.

8. The system of claim 2, wherein stimuli delivered via the one or more of the plurality of ICSs are bi-phasic and include a positive phase of a pulse and a negative phase of the pulse.

9. The system of claim 2, wherein the plurality of ICSs are each configured to monitor accuracy of ICS current and wherein the MCU is configured to monitor total energy delivered to a patient, wherein in response to the total energy meeting a predefined threshold, stimulus delivery is aborted.

10. The system of claim 2, further comprising:
a front-end amplifier unit (FEAU) that operably couples to the MCU, wherein the FEAU is coupled to the multi-electrode cap including a plurality of electroencephalogram (EEG) electrodes; and
a plurality of additional monitoring electrodes measuring physiology of a patient, the plurality of additional monitoring electrodes including electrocardiogrph (ECG) and electromyogram (EMG) electrodes.

11. The system of claim 10, wherein the FEAU is configured to protect the plurality of EEG electrodes during stimulation and to reduce EEG reading recovery time delay.

12. The system of claim 10 further comprising an operator computer communicatively and operably coupled to the MCU and the multi-electrode cap, wherein the operator computer is configured to display a GUI through which one or more indications are displayed, including status of stimulation signal, and controls are provided to specify nature, timing, and onset of neurostimulation, wherein the GUI further displays physiological signals of ECG, EEG, and EMG data recorded from the FEAU.

13. A method, comprising:
delivering current pulses to one or more stimulation targets of a patient, wherein the current pulses delivered are variable in position and dosimetry based on the one or more stimulation targets and patient-specific anatomy.

14. The method of claim 13, further comprising:
defining the one or more stimulation targets of the patient;
determining a multi-electrode configuration for a multichannel individualized stimulation therapy (MIST) system;
charging a plurality of batteries of the MIST system, wherein the plurality of batteries comprises a main control unit (MCU) battery and a plurality of current sources batteries including one current source battery for each of a plurality of isolated current sources (ICSs); and
in response to a determination that the plurality of batteries are charged, operating the MIST system to deliver stimulus to the one or more stimulation targets of the patient via one or more of the plurality of ICSs.

15. The method of claim 14, wherein each of the plurality of ICSs is configured to deliver current to the patient through a pair of stimulus terminals, wherein the pair of stimulus terminals is selectively chosen based on the multi-electrode configuration.

16. The method of claim 14, wherein determining the multi-electrode configuration comprises:
applying a computational optimization method for a computed electric field to a three-dimensional (3D) model of a head of the patient generated from structural data of the patient;
determining electrode current distribution based on the computational optimization method; and
mapping current sources to the electrode current distribution.

17. The method of claim 16, wherein mapping the current sources to the electrode current distribution comprises defining positions of each of a plurality of electrodes of a multi-electrode cap and defining pairs of electrodes based on the electrode current distribution.

18. The method of claim 17, wherein mapping the current sources further comprises assigning each of the one or more of the plurality of ICSs to one pair of electrodes of the plurality of electrodes, wherein a first electrode of the one pair is assigned to a first terminal of an assigned current source and a second electrode of the one pair is assigned to a second terminal of the assigned current source, and wherein operating the MIST system to deliver stimulus to the patient comprises delivering current across each of the pairs of electrodes.

19. The method of claim 18, wherein delivering current across each of the pairs of electrodes defines one or more current paths, wherein the one or more current paths and the positions of each of the plurality of electrodes are specified based on one or more stimulation targets, wherein the plurality of ICSs are configured to produce stimulation voltage with a total accumulate stimulus current that is formed in the one or more current paths between electrodes.

20. The method of claim 14, wherein the MIST system is operated to deliver stimulus to the patient for treating one or more of a neurological, psychological, and psychiatric disorder.
